(19) United States
(12) Patent Application Publication
(10) Pub. No.: US 2012/0258940 A1
(43) Pub. Date: Oct. 11, 2012

(54) METHOD FOR TREATING HAEMATOLOGICAL CANCERS

(76) Inventors: Giordano Caponigro, Foxborough, MA (US); Diana Graus Porta, Basel (CH); Yao Yao, Shanghai (CN); Vito Guagnano, Basel (CH)

(21) Appl. No.: 13/516,093

(22) PCT Filed: Dec. 17, 2010
(86) PCT No.: PCT/US10/60956

§ 371 Date: Jun. 14, 2012

Related U.S. Application Data
(60) Provisional application No. 61/287,831, filed on Dec. 18, 2009.

(51) Int. Cl. A61K 31/573; A61P 35/00

(52) U.S. Cl. 514/171

(57) ABSTRACT

The present invention relates to a combination which comprises (a) a FGFR inhibitor and (b) a glucocorticoid receptor modulator, or a pharmaceutical acceptable salt thereof; the use of such a combination for the preparation of a medicament for the treatment of haematological cancers; a commercial package or product comprising such a combination; and to a method of treatment of a warm-blooded animal, especially a human.

The present invention relates to a combination which comprises (a) a FGFR inhibitor and (b) a glucocorticoid receptor modulator, or a pharmaceutical acceptable salt thereof; the use of such a combination for the preparation of a medicament for the treatment of haematological cancers; a commercial package or product comprising such a combination; and to a method of treatment of a warm-blooded animal, especially a human.

### BACKGROUND OF THE INVENTION

Fibroblast growth factor receptors (FGFRs) comprise a subfamily of receptor tyrosine kinases (RTKs) that are master regulators of a broad spectrum of biological activities, including development, metabolism, angiogenesis, apoptosis, proliferation and migration. Due to their broad impact, FGFRs and other RTKs are highly regulated and normally only basally active.

Epidemiological studies have reported genetic alterations and/or abnormal expression of FGFs/FGFRs in human cancers: translocation and fusion of FGFR1 to other genes resulting in constitutive activation of FGFR1 kinase is responsible for 8 μl myeloproliferative disorder (MacDonald D & Cross N C, Pathobiology 74:81-8 (2007)). Gene amplification and protein over-expression have been reported for FGFR1, FGFR2 and FGFR4 in breast tumors (Adnane J at al., Oncogene 6:659-63 (1991); Jaakkola S at al., Int. J. Cancer 54:378-82 (1993); Penault-Llorca F et al., Int. J. Cancer 61: 170-6 (1995); Reis-Filho J S et al., Clin. Cancer Res. 12:6652-62 (2006)). Somatic activating mutations of FGFR2 are known in gastric Wang J H et al., Cancer Res. 61:3541-3 (2001)) and endometrial cancers (Pollock P M et al., Oncogene (May 21, 2007)). Recurrent chromosomal translocations of 4p16 into the immunoglobuling heavy chain switch region at 14q32 result in deregulated over-expression of FGFR3 in multiple myeloma (Chesi M et al., Nature Genetics 16:260-264 (1997); Chesi M et al., Blood 97:729-736 (2001)) and somatic mutations in specific domains of FGFR3 leading to ligand-independent constitutive activation of the receptor have been identified in urinary bladder carcinomas and multiple myelomas (Cappellen D et al., Nature Genetics 23:18-20 (1999); Billerey C et al., Am. J. Pathol. 158(6):1955-9 (2001); van Rhijn B W G et al., Eur. J. Hum. Genet. 10: 819-824 (2002); Ronchetti C et al., Oncogene 20: 3553-3562 (2001)).

Multiple myeloma is an incurable malignancy of terminally differentiated B cells, characterized by clonal expansion of plasma cells in the bone marrow. Approximately 15% to 20% of MM cases involved t(4; 14) (p16.3; q32.3) translocation, resulting in the dysregulated expression of 2 putative oncogenes, MMSET and FGFR3 (Chesi M et al., Nat. Genet. 16: 260-264 (1991)). This translocation event is associated with a particularly poor prognosis, marked by a substantially shortened survival following either conventional or high-dose chemotherapy (Morealu P et. Al., Blood 100: 1579-1583 (2002)). Roughly 10% of these patients further acquire activating mutations in FGFR3, an additional adverse prognostic factor (Intini D et al., Br. J. Haematol., 114: 362-364 (2001)). Inhibition of FGFR3 activity inhibits tumor growth in cell lines and animal models of FGFR3-associated MM, supporting its therapeutic relevance (Trudel S et al., Blood 107: 4039-4046 (2006); Xin X et al., Clin. Can. Res., 12: 4908-4915 (2006)).

Glucocorticoids (GCs) are steroid hormones produced by the adrenal glands after cytokine stimulation of the hypothalamus-pituitary-adrenal axis. All natural steroid hormones share a common multi-ring structure and have additional chemical groups bound to the steroid nucleus that confer specificity to their actions. Dexamethasone (Dex), a synthetic steroidal glucocorticoid, is a multiring structure with an added fluorine atom (Clark R. D. Cur. Top. Med. Chem., 8: 813-838 (2008)). Fluorine increases drug potency by slowing metabolism and also increases the affinity of Dex for its receptor, the glucocorticoid receptor (GR) (Tannock I. F., The Basic Science of Oncology, Ed 2, p. 420. Toronto: McGraw-Hill, Inc., 1992). GR is a member of the nuclear receptor protein family. In the absence of GC, GR resides in the cytosol complexed with a variety of proteins including the heat shock protein 90 (hsp90), the heat shock protein 70 (hsp70) and the immuniphilin FKBP52 (FK506-binding protein 52). Dexamethasone or the endogenous glucocortiod hormone cortisol diffuses through the cell membrane into the cytoplasm and binds to GR resulting in release of the heat shock proteins and translocation of the GC-GR complex into the nucleus. In the nucleus, GR can form homodimmers and bind to Glucocorticoid Responsive Element (GRE) on DNA, resulting in transactivation. Alternatively, GR can heterodimmerize with other transcription factors such as NFkB and AP-1 to prevent transcripton of their target genes, a phenomenon termed transrepression (Hayashi R. et al., Eur. J. Pharmacol., 500: 51-62, (2004)). Through GR, GCs are involved in the regulation of a variety of biological processes, including immune responses, metabolism, cell growth and proliferation, development, and reproduction.

As early as in the 1940's glucocorticoids were found effective in inhibiting the growth of leukemic tumors, and subsequently introduced as the first line drug in the treatment of childhood acute lymphoblastic leukemia (ALL). Later studies indicated that GCs are potent inducers of apoptosis in thymocytes and leukemic cells, which provided the basis for their clinical usefulness. Today GCs constitute central components in the treatment of various hematological malignancies such as ALL, multiple myeloma (MM), chronic lymphocytic leukemia (CLL) and non-Hodgkin's lymphoma, besides their wide use as anti-inflammatory drugs in autoimmune and inflammatory diseases (Sionov R V et al., Cel. Cycl., 5:10: 1017-1026 (2006)).

### SUMMARY OF INVENTION

It has been surprisingly found that modulators of glucocorticoid receptor are able to potentiate the antiproliferative activity of FGFR inhibitors. It is therefore an object of present invention to provide for a medicament to improve medication of haematological cancers. The term “haematological cancers” in this context includes haematological malignancies. Hematological malignancies are the types of cancer that affect blood, bone marrow, and lymph nodes.

Preferably, the haematological cancers as referred to herein, are multiple myelomas (MM). In particular, such multiple myelomas are multiple myeloma with t (4,14) chromosomal translocation and/or FGFR3 over-expression.

The present invention reports that a combination comprising (a) an FGFR inhibitor and (b) a modulator of glucocorticoid receptor, can produce a therapeutic effect which is greater than that obtainable by administration of a therapeutically effective amount of either an FGFR inhibitor, or a modulator of glucocorticoid receptor alone. Furthermore the present invention reports that a combination comprising (a) an FGFR inhibitor and (b) a modulator of glucocorticoid receptor produces a strong synergistic effect.

The present invention also pertains to a combination for simultaneous, separate or sequential use, such as a combined preparation or a pharmaceutical fixed combination. A fixed combination refers to both active ingredients present in one dosage form, e.g. in one tablet or in one capsule. The combination of the present invention comprises (a) an FGFR inhibitor and (b) a modulator of glucocorticoid receptor, in which the active ingredients (a) and (b) are present in each case in free form or in the form of a pharmaceutically acceptable salt, and optionally at least one pharmaceutically acceptable carrier.

The term “a combined preparation” or “combination”, as used herein defines especially a “kit of parts” in the sense that the combination partners (a) and (b) as defined herein can be dosed independently of each other or by use of different fixed combinations with distinguished amounts of the combination partners (a) and (b), i.e. simultaneously or at different time points. The parts of the kit of parts can then, e.g. be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to age, sex, body weight, etc. of the patients. Preferably, there is at least one beneficial effect, e.g. a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The term “treatment” comprises the administration of the combination partners to a warm-blooded animal, preferably to a human being, in need of such treatment with the aim to cure the disease or to have an effect on disease regression or on the delay of progression of a disease.

Therefore, present invention relates to a combination of (a) an FGFR inhibitor and (b) a modulator of glucocorticoid receptor or, respectively, a pharmaceutically acceptable salt thereof.

A further embodiment of this invention provides a combination comprising a quantity, which is jointly therapeutically effective against haematological cancers comprising the combination partners (a) and (b). Thereby, the combination partners (a) and (b) can be administered together, one after the other or separately in one combined unit dosage form or in two separate unit dosage forms. The unit dosage form may also be a fixed combination.

The combinations according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application. In one embodiment of the invention, one or more of the active ingredients are administered orally.

A further embodiment relates to the use of the inventive combination for treating haematological cancers. A further embodiment relates to the use of present combination for the manufacture of a medicament for treating haematological cancers. A further embodiment relates to a method of treating haematological cancers with a combination of an FGFR inhibitor and a modulator of glucocorticoid receptor or, respectively, a pharmaceutically acceptable salt thereof. A further embodiment of present invention relates to a commercial package comprising a combination according to the invention described herein, together with instructions for simultaneous, separate or sequential use thereof in the treatment of haematological cancers.

A further embodiment of present invention relates to the use of Compound A for the preparation of a combination according to present invention, i.e. for the preparation of a combination with a modulator of glucocorticoid receptor, in particular with dexamethasone.

A number of FGFR inhibitors, with high or medium selectivity towards FGFRs has been disclosed.

WO 06/000420 and WO 07/071,752 disclose a group of compounds with high selectivity towards FGFRs. Both publications are hereby enclosed into the present application by reference.

Examples for FGFR inhibitors (a) according to the invention are compounds of formula IA,

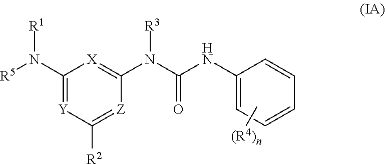

wherein

two of X, Y and Z are N (nitrogen), the third is CH or N (preferably Y and Z are N and Z is CH); and

wherein either

R¹ is phenyl that is substituted by hydroxy, phenyl-C₁-C₇-alkyloxy, piperazin-1-yl or 4-(phenyl-C₁-C₇-alkyl)-piperazin-1-yl; or phenyl that is substituted by (i) halo or C₁-C₇-alkoxy and in addition (ii) by hydroxy, phenyl-C₁-C₇-alkyloxy, N-mono- or N,N-di-(C₁-C₇-alkyl)-amino-C₁-C₇-alkyl, pyrrolidino-C₁-C₇-alkoxy, 1-(C₁-C₇-alkyl)-piperidin-4-yl, morpholino-C₁-C₇-alkoxy, thiomorpholino-C₁-C₇-alkoxy, piperazin-1-yl, 4-(phenyl-C₁-C₇-alkyl)-piperazin-1-yl, 4-(C₁-C₇-alkyl)-piperazin-1-yl, [4-(C₁-C₇-alkyl)-piperazin-1-yl]-C₁-C₇-alkyl, N-mono- or N,N-di-(C₁-C₇-alkyl)-amino-C₁-C₇-alkyl, N-mono- or N,N-di-(C₁-C₇-alkyl)-amino-C₁-C₇-alkoxy, [4-(C₁-C₇-alkyl)-piperazin-1-yl]-C₁-C₇-alkoxy, [4-(C₁-C₇-alkyl)-piperazin-1-yl]-carbonyl;

R² is hydrogen, C₁-C₇-alkyl, C₁-C₇-alkoxy or halo;

R³ is hydrogen, C₁-C₇-alkyl or phenyl-C₁-C₇-alkyl,

each R⁴ is, independently of the others, C₁-C₇-alkyl, halo-C₁-C₇-alkyl, halo or C₁-C₇-alkoxy,

and n is 0, 1, 2, 3, 4 or 5;

or

R¹ is phenyl that is substituted by hydroxy, phenyl-C₁-C₇-alkyloxy, piperazin-1-yl, 4-(phenyl-C₁-C₇-alkyl)-piperazin-1-yl; N-mono- or N,N-di-(C₁-C₇-alkyl)-amino-C₁-C₇-alkyl, pyrrolidino-C₁-C₇-alkoxy, 1-(C₁-C₇-alkyl)-piperidin-4-yl, morpholino-C₁-C₇-alkoxy, thiomorpholino-C₁-C₇-alkoxy, 4-(C₁-C₇-alkyl)-piperazin-1-yl, [4-(C₁-C₇-alkyl)-piperazin-1-yl]-C₁-C₇-alkyl, N-mono- or N,N-di-(C₁-C₇-alkyl)-amino-C₁-C₇-alkyl, N-mono- or N,N-di-(C₁-C₇-alkyl)-amino-C₁-C₇-alkoxy, [4-(C₁-C₇-alkyl)-piperazin-1-yl]-C₁-C₇-alkoxy, [4-(C₁-C₇-alkyl)-piperazin-1-yl]-carbonyl; or phenyl that carries one of the substitutents mentioned so far in the present paragraph and in addition a substituent selected from halo and C₁-C₇-alkoxy;

R² is hydrogen, C₁-C₇-alkyl, C₁-C₇-alkoxy or halo;

R³ is hydrogen, C₁-C₇-alkyl or phenyl-C₁-C₇-alkyl,

R⁵ is hydrogen (preferred), C₁-C₇-alkyl or phenyl-C₁-C₇-alkyl,
and
either n is 3, 4 or 5 and R⁴ is selected from C₁-C₇-alkyl, C₁-C₇-alkoxy and halo, with the proviso that at least one of each of C₁-C₇-alkyl, C₁-C₇-alkoxy and halo is present;
or n is 2 and one R⁴ is halo-C₁-C₇-alkyl, the other R⁴ is C₁-C₇-alkoxy;
or n is 3, 4 or 5 and R⁴ is selected from halo, iodo and C₁-C₇-alkoxy, with the proviso that at least one of each of halo, iodo and C₁-C₇-alkoxy, is present;
or n is 3, 4 or 5 and R⁴I selected from halo, halo-C₁-C₇-alkyl and C₁-C₇-alkoxy, with the proviso that at least one of each of halo, halo-C₁-C₇-alkyl and C₁-C₇-alkoxy is present;

or

Y and Z are N (nitrogen) and X is CH,

wherein either

R¹ is 3-pyridyl which is monosubstituted by N—C₁-C₇-alkyl-piperazin-1-yl,

R² is hydrogen,

R³ is hydrogen,

each R⁴ is, independently of the others, C₁-C₇-alkyl, halo-C₁-C₇-alkyl, halo or C₁-C₇-alkoxy,

R⁵ is hydrogen

and n is 1, 2, 3, 4 or 5;

or

a compound of the formula IA wherein R¹ is 4-(2-morpholin-4-yl-ethoxy)-phenylamino, R² is hydrogen, R³ is hydrogen, R⁴ is 2- and 6-chloro and 3- and 5-methoxy, n is 4, R⁵ is hydrogen, Y and Z are N and X is CH;

or

a compound of the formula IA wherein R¹ is 3-(4-methyl-piperazin-1-ylmethyl)-phenylamino, R² is hydrogen, R³ is methyl, R⁴ is 2- and 6-chloro and 3- and 5-methoxy, n is 4, R⁵ is hydrogen, Y and Z are N and X is CH,

or

a compound of the formula IA wherein R¹ is 3-(4-ethyl-piperazin-1-yl)-phenylamino, R² is hydrogen, R³ is methyl, R⁴ is 2- and 6-chloro and 3- and 5-methoxy, n is 4, R⁵ is hydrogen, Y and Z are N and X is CH,

or

a compound of the formula IA wherein R¹ is 4-(2-morpholin-4-yl-ethoxy)-phenylamino, R² is hydrogen, R³ is methyl, R⁴ is 2- and 6-chloro and 3- and 5-methoxy, n is 4, R⁵ is hydrogen, Y and Z are N and X is CH,

or

a compound of the formula IA wherein R¹ is 4-(1-ethyl-piperidin-4-yl)-phenylamino, R² is hydrogen, R³ is methyl, R⁴ is 2- and 6-chloro and 3- and 5-methoxy, n is 4, R⁵ is hydrogen, Y and Z are N and X is CH,

or

a compound of the formula IA wherein R¹ is 4-(4-ethyl-pipeazin-1-yl)-phenylamino, R² is hydrogen, R³ is ethyl, R⁴ is 2- and 6-chloro and 3- and 5-methoxy, n is 4, R⁵ is hydrogen, Y and Z are N and X is CH, and/or

or

a compound of the formula IA wherein R¹ is 4-(4-ethyl-piperazine-1-carbonyl)-phenylamino, R² is hydrogen, R³ is methyl, R⁴ is 2- and 6-chloro and 3- and 5-methoxy, n is 4, R⁵ is hydrogen, Y and Z are N and X is CH;

or mixtures of two or more compounds of the formula IA;

or a salt, a prodrug, an N-oxide and or an ester thereof.

Examples of compounds according to formula IA are:

1-[6-(4-benzyloxy-phenylamino)-pyrimidin-4-yl]-3-(2,6-dichloro-3,5-dimethoxy-phenyl)-1-methyl-urea, 3-(2,6-dichloro-3,5-dimethoxy-phenyl)-1-[6-(4-hydroxy-phenylamino)-pyrimidin-4-yl]-1-methyl-urea, 1-{6-[4-(4-benzyl-piperazin-1-yl)-phenylamino]-pyrimidin-4-yl}-3-(2,6-dichloro-3,5-dimethoxy-phenyl)-1-methyl-urea, 3-(2,6-dichloro-3,5-dimethoxy-phenyl)-1-methyl-1-[6-(4-piperazin-1-yl-phenylamino)-pyrimidin-4-yl]-urea, 3-(2,6-dichloro-3,5-dimethoxy-phenyl)-1-{6-[2-fluoro-4-(2-pyrrolidin-1-yl-ethoxy)-phenylamino]-pyrimidin-4-yl}-1-methyl-urea, 3-(2,6-dichloro-3,5-dimethoxy-phenyl)-1-{6-[4-(4-ethyl-piperazin-1-yl)-2-methoxy-phenylamino]-pyrimidin-4-yl}-1-methyl-urea, 3-(2,6-dichloro-3,5-dimethoxy-phenyl)-1-{6-[4-(4-ethyl-piperazin-1-yl)-3-fluoro-phenylamino]-pyrimidin-4-yl}-1-methyl-urea, 3-(5-methoxy-3-trifluoromethyl-phenyl)-1-{6-[3-chloro-4-(4-ethyl-piperazin-1-yl)-phenylamino]-pyrimidin-4-yl}-1-methyl-urea, 1-{6-[2-chloro-4-(4-ethyl-piperazin-1-yl)-phenylamino]-pyrimidin-4-yl}-3-(2,6-dichloro-3,5-dimethoxy-phenyl)-1-methyl-urea and 3-(2,6-dichloro-3,5-dimethoxy-phenyl)-1-{6-[4-(4-ethyl-piperazin-1-yl)-2-fluoro-phenylamino]-pyrimidin-4-yl}-1-methyl-urea; or a salt, a prodrug, an N-oxide and or an ester thereof. Further compounds of the formula IA are 1-(2-chloro-3,5-dimethoxy-6-methyl-phenyl)-3-{6-[4-(4-ethyl-piperazin-1-yl)-phenylamino]-pyrimidin-4-yl}-urea, 3-(2-chloro-3,5-dimethoxy-6-methyl-phenyl)-1-{6-[4-(4-ethyl-piperazin-1-yl)-phenylamino]-pyrimidin-4-yl}-1-methyl-urea, 3-(2-chloro-3,5-dimethoxy-6-methyl-phenyl)-1-{6-[4-(2-dimethylamino-ethoxy)-phenylamino]-pyrimidin-4-yl}-1-methyl-urea, 3-(2-chloro-3,5-dimethoxy-6-methyl-phenyl)-1-methyl-(6-{4-[2-(4-methyl-piperazin-1-yl)-ethoxy]-phenylamino}-pyrimidin-4-yl)-urea, 3-(2-chloro-6-iodo-3,5-dimethoxy-phenyl)-1-{6-[4-(4-ethyl-piperazin-1-yl)-phenylamino}-pyrimidin-4-yl]-1-methyl-urea, 3-(2-chloro-3,5-dimethoxy-6-methyl-phenyl)-1-{6-[4-(4-isopropyl-piperazin-1-yl)-phenylamino]-pyrimidin-4-yl}-1-methyl-urea, 3-(2-chloro-3,5-dimethoxy-6-methyl-phenyl)-1-[6-(3-dimethylaminomethyl-phenylamino)-pyrimidin-4-yl]-1-methyl-urea, 3-(2-chloro-3,5-dimethoxy-6-methyl-phenyl)-1-{6-[3-(4-ethyl-piperazin-1-yl)-phenylamino]-pyrimidin-4-yl}-1-methyl-urea, 3-(2-chloro-3,5-dimethoxy-6-methyl-phenyl)-1-methyl-1-{6-[4-(2-pyrrolidin-1-yl-ethoxy)-phenylamino]-pyrimidin-4-yl}-1-methyl-urea, 3-(2-chloro-3,5-dimethoxy-6-methyl-phenyl)-1-methyl-1-{6-[3-fluoro-4-(2-pyrrolidin-1-yl-ethoxy)-phenylamino]-pyrimidin-4-yl}-1-methyl-urea, 3-(2,4-dichloro-5-methoxy-3-trifluoromethyl-phenyl)-1-{6-[4-(4-ethyl-piperazin-1-yl)-phenylamino]-pyrimidin-4-yl}-1-methyl-urea and 3-(5-methoxy-3-trifluoromethyl-phenyl)-1-{6-[4-(4-ethyl-piperazin-1-yl)-phenylamino]-pyrimidin-4-yl}-1-methyl-urea; or a salt, a prodrug, an N-oxide and or an ester thereof.

Further compounds of the formula IA are: 1-(2,6-dichloro-3,5-dimethoxy-phenyl)-3-{6-[4-(2-morpholin-4-yl-ethoxy)-phenylamino]-pyrimidin-4-yl}-urea, 3-(2,6-dichloro-3,5-dimethoxy-phenyl)-1-methyl-1-{6-[3-(4-methyl-piperazin-1-ylmethyl)-phenylamino]-pyrimidin-4-yl}-urea, 3-(2,6-dichloro-3,5-dimethoxy-phenyl)-1-{6-[3-(4-ethyl-piperazin-1-yl)-phenylamino]-pyrimidin-4-yl}-1-methyl-urea, 3-(2,6-dichloro-3,5-dimethoxy-phenyl)-1-methyl-1-{6-[4-(2-morpholin-4-yl-ethoxy)-phenylamino]-pyrimidin-4-yl}-urea, 3-(2,6-dichloro-3,5-dimethoxy-phenyl)-1-{6-[4-(1-ethyl-piperidin-4-yl)-phenylamino]-pyrimidin-4-yl}-1-methyl-urea, 3-(2,6-dichloro-3,5-dimethoxy-phenyl)-1-ethyl-1-{6-[4-(4-ethyl-piperazin-1-yl)-phenylamino]-pyrimidin-4-yl}-urea; and 3-(2,6-dichloro-3,5-dimethoxy-phenyl)-1-{6-[4-(4-ethyl-piperazine-1-carbonyl)-phenylamino]-pyrimidin-4-yl}-1-methyl-urea; or a salt, a prodrug, an N-oxide and or an ester thereof.

Further compounds of the formula IA are: 3-(2,6-dichloro-3,5-dimethoxy-phenyl)-1-{6-[6-(4-ethyl-piperazin-1-yl)-pyridin-3-ylamino]-pyrimidin-4-yl}-1-methyl-urea; and 3-(2,6-dichloro-3,5-dimethoxy-phenyl)-1-{6-[6-(4-isopropyl-piperazin-1-yl)-pyridin-3-ylamino]-pyrimidin-4-yl}-1-methyl-urea, or a salt, a prodrug, an N-oxide and or an ester thereof.

The expression “FGFR inhibitor” as used herein hence includes the compounds of formula IA. In particular, it includes, 3-(2,6-dichloro-3,5-dimethoxy-phenyl)-1-{6-[4-(4-ethyl-perpazin-1-yl)-phenylamino]-pyrimidin-4-yl}-1-methyl urea, or a pharmaceutically acceptable salt thereof, referenced herein as Compound A. Compound A is a small molecular mass inhibitor that is highly selective for FGFR1-4 (example 145 of WO2006/000420) in two t(4; 14) multiple myeloma cell lines, KMS-11 and OPM-2, harboring gain-of-function mutation, FGFR3-Y373C and FGFR3-K650E, respectively.

Another example for an FGFR inhibitor is the compound of example 109 of WO02/22598, namely 4-amino-5-fluoro-3-[6-(4-methylpiperazin-yl)-1H-benzimidazol-2-yl]quinolin-2(1H)-one, herein referred to as Compound B.

Further FGFR inhibitors have been disclosed, for example Brivanib (Compound C) is disclosed as (2R)-1-[4-(4-Fluoro-2-methyl-1H-indol-5-yloxy)-5-methyl-pyrrolo[2,1-f][1,2,4]triazin-6-yloxy]-propan-2-ol in Example 15 of WO 2004/009784. Compound D is disclosed as (R)-N-(3-(3,5-Dimethoxy-phenyl)-ethyl)-1H-pyrazol-3-yl)-4-(3,4-dimethyl-piperazin-1-yl)benzamide in Example 1(b) of WO 2009/153592. Another FGFR inhibitor is the compound of example 14 of WO 07/071,752, namely 3-(2,6-Dichloro-3,5-dimethoxy-phenyl)-1-methyl-1-[6-(4-piperazin-1-yl-phenylamino)-pyrimidin-4-yl]-urea (compound F).

PD173074 (compound E), 1-tert-Butyl-3-[6-(3,5-dimethoxy-phenyl)-2-(4-diethylamino-butylamino)-pyrido[2,3-d]pyrimidin-7-yl]-urea, is disclosed as an FGF-R specific inhibitor from Parke Davis (Mohammadi et al., 1998, EMBO J. 17: 5896-5904).

The expression “modulator of glucocorticoid receptor” as used herein refers to a class of steroid hormones, naturally occurring or synthetically made, that bind to Glucocorticoid receptor to modulates its function. Preferably a GR modulating agent is a GR activating agent, including but not limiting to Dexamethasone and Halomethasone. Commonly and preferably used GR modulating agent is Dexamethasone or Halomethasone.

In a preferred embodiment, the modulator of glucocorticoid receptor is dexamethasone, a compound described for instance by Clark R. D. in Cur. Top. Med. Chem., 8: 813-838 (2008)).

In another preferred embodiment, the modulator of glucocorticoid receptor is halometasone.

Thus the present invention relates to a combination for simultaneous, separate or sequential use, such as a combined preparation or a pharmaceutical fixed combination, which comprises (a) an FGFR inhibitor and (b) a modulator of glucocorticoid receptor, in which the active ingredients (a) and (b) are present in each case in free form or in the form of a pharmaceutically acceptable salt, and optionally at least one pharmaceutically acceptable carrier.

In one embodiment, the FGFR inhibitor is selected from a group consisting of:
(1) 3-(2,6-dichloro-3,5-dimethoxy-phenyl)-1-{6-[4-(4-ethyl-perpazin-1-yl)-phenylamino]-pyrimidin-4-yl}-1-methyl urea;
(2) 4-amino-5-fluoro-3-[6-(4-methylpiperazin-1-yl)-1H-benzimidazol-2-yl]quinolin-2(1H)-one;
(3) (2R)-1-[4-(4-Fluoro-2-methyl-1H-indol-5-yloxy)-5-methyl-pyrrolo[2,1-f][1,2,4]triazin-6-yloxy]-propan-2-ol;
(4) (R)-N-(3-(3,5-Dimethoxy-phenyl)-ethyl)-1H-pyrazol-3-yl)-4-(3,4-dimethyl-piperazin-1-yl)benzamide;
(5) 1-tert-Butyl-3-[6-(3,5-dimethoxy-phenyl)-2-(4-diethylamino-butylamino)-pyrido[2,3-d]pyrimidin-7-yl]-urea; and
(6) 3-(2,6-Dichloro-3,5-dimethoxy-phenyl)-1-methyl-1-[6-(4-piperazin-1-yl-phenylamino)-pyrimidin-4-yl]-urea

In free form, complex form or a pharmaceutically acceptable salt thereof.

In one embodiment, the modulator of glucocorticoid receptor is selected from a group consisting of (1) dexamethasone and (2) halometasone.

In one preferred embodiment, the FGFR inhibitor is selected from a group consisting of:
(1) 3-(2,6-dichloro-3,5-dimethoxy-phenyl)-1-{6-[4-(4-ethyl-perpazin-1-yl)-phenylamino]-pyrimidin-4-yl}-1-methyl urea; and
(2) 4-amino-5-fluoro-3-[6-(4-methylpiperazin-1-yl)-1H-benzimidazol-2-yl]quinolin-2(1H)-one

In free form, complex form or a pharmaceutically acceptable salt thereof,

In one preferred embodiment, the modulator of glucocorticoid receptor is dexamethasone.

Therefore, a very preferred embodiment of present invention relates to a combination of 3-(2,6-Dichloro-3,5-dimethoxy-phenyl)-1-{6-[4-(4-ethyl-piperazin-1-yl)-phenylamino]-pyrimidin-4-yl}-1-methyl-urea in free form, complex form or, respectively, a pharmaceutically acceptable salt thereof and dexamethasone

An again very preferred embodiment of present invention relates to a combination of 4-amino-5-fluoro-3-[6-(4-methylpiperazin-1-yl)-1H-benzimidazol-2-yl]quinolin-2(1H)-one or, respectively, a pharmaceutically acceptable salt thereof and dexamethasone

A further embodiment relates to the use of this inventive combination for treating haematological cancers. A further embodiment relates to the use of such combination for the manufacture of a medicament for treating haematological cancers. In one preferred embodiment, the haematological cancer is multiple myeloma.

A further embodiment relates to a method of treating haematological cancers, with a combination of 3-(2,6-Dichloro-3,5-dimethoxy-phenyl)-1-{6-[4-(4-ethyl-piperazin-1-yl)-phenylamino]-pyrimidin-4-yl}-1-methyl-urea and dexamethasone or, respectively, a pharmaceutically acceptable salt thereof.

In one aspect the invention provides a use of 3-(2,6-dichloro-3,5-dimethoxy-phenyl)-1-{6-[4-(4-ethyl-perpazin-1-yl)-phenylamino]-pyrimidin-4-yl}-1-methyl urea for the manufacture of a medicament to be used in combination with dexamethasone or with Sicorten for the treatment of haematological cancers, preferably multiple myeloma.

### EXAMPLES

The following examples illustrate the invention described above, but are not, however, intended to limit the scope of the invention in any way. Other test models known as such to the person skilled in the pertinent art can also determine the beneficial effects of the claimed invention.

### BRIEF DESCRIPTION OF THE DRAWINGS

FIG. 1 is a graph describing the synergistic effect between Compound A and dexamethasone observed in multiple myeloma cell line KMS-11:

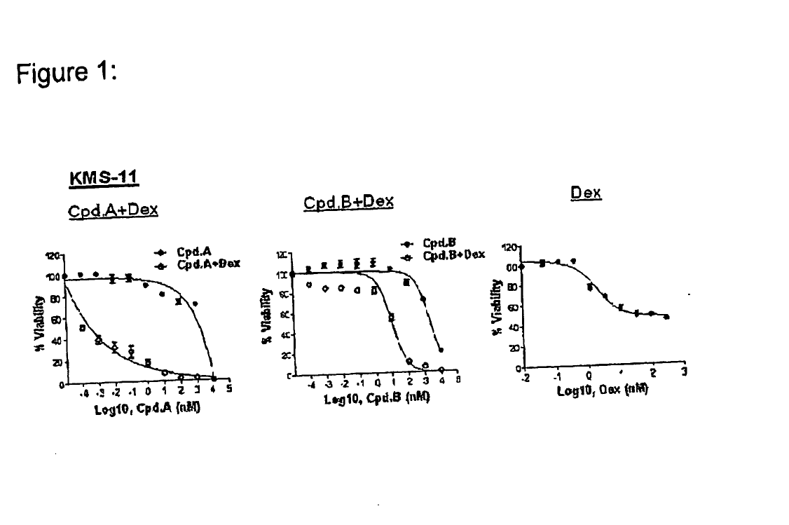

KMS-11 cells were treated with Compound A and dexamethasone or dexamethasone alone, respectively, for 72 hours; the comparison to the combination of Compound B and dexamethasone is shown.

FIG. 2 is a graph describing the synergistic effect between Compound A and dexamethasone observed in multiple myeloma cell line OPM-2:

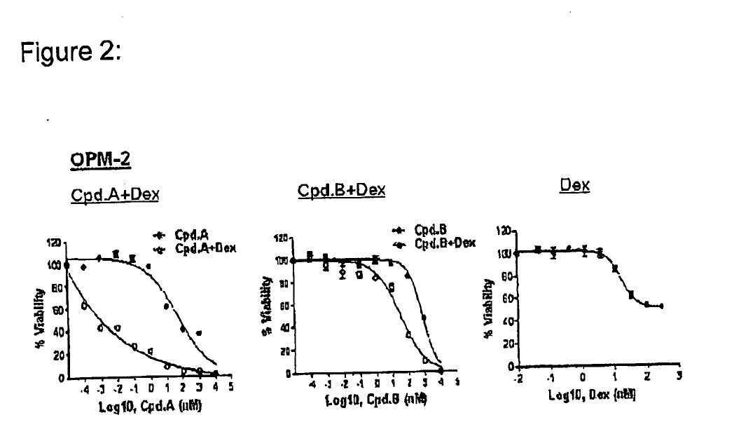

OPM-2 cells were treated with Compound A and dexamethasone or dexamethasone alone, respectively, for 72 hours; the comparison to the combination of Compound B and dexamethasone is shown.

Cell viability was measured by CellTiter GLO. For the combination treatments in FIGS. 1 and 2, the Y axes were cell viability was normalized to the wells treated with dexamethasone alone.

FIGS. 3a and 3b are graphs describing the synergistic effect between Compound A and two synthetic glucocorticoids, dexamethasone and Sicorten, observed in multiple myeloma cell lines KMS-11 and OPM-2.

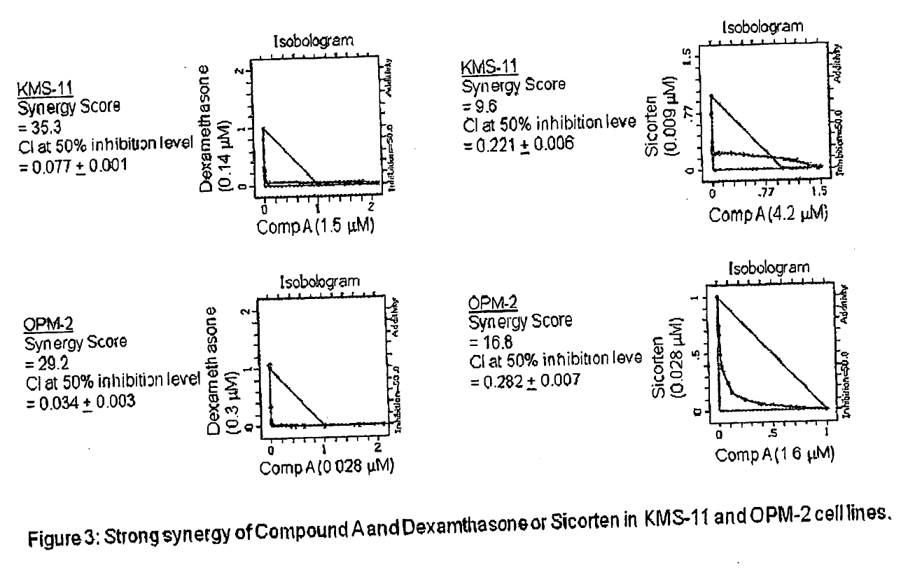

FIGS. 4a and 4b are graphs describing the synergistic effect between Compound B and two synthetic glucocorticoids, dexamethasone and Sicorten, observed in multiple myeloma cell lines KMS-11 and OPM-2.

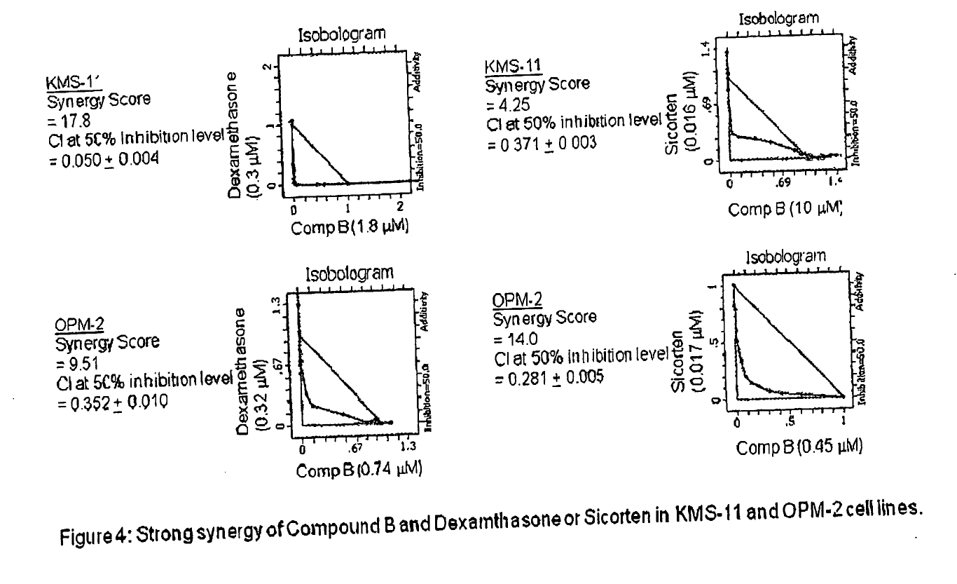

FIGS. 5a and 5b are graphs describing the synergistic effect between Compound E (PD173074) and one synthetic glucocorticoid and dexamethasone in multiple myeloma cell lines KMS-11 and OPM-2.

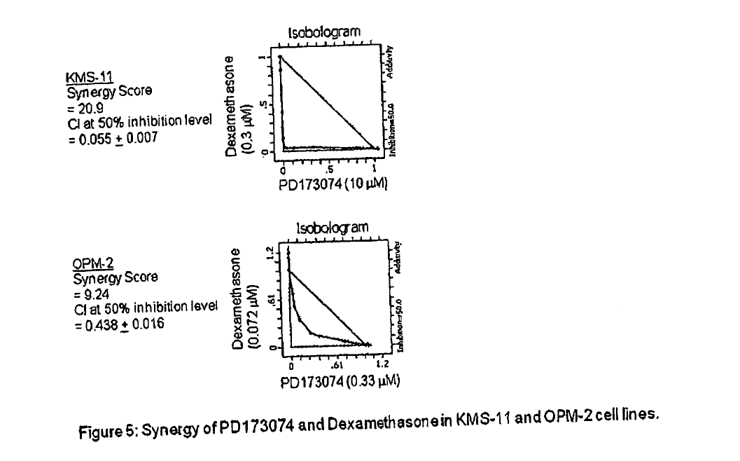

TABLE 1
Summary of combination effect with Compound A and
dexamethasone in myeloma cell line KMS-11, in
comparison to Compound B and dexamethasone.
KMS-11

| | CI of A + 100 nM | | CI of B + 100 nM |
|---|---|---|---|
| Cpd. A (nM) | Dex | Cpd. B (nM) | Dex |
| 0.0001 | 6.10E−02 | 0.0001 | 2.71E−01 |
| 0.001 | 2.20E−02 | 0.001 | 1.72E−01 |
| 0.01 | 9.00E−03 | 0.01 | 1.73E−01 |
| 0.1 | 5.00E−03 | 0.1 | 1.32E−01 |
| 1 | 1.00E−03 | 1 | 1.23E−01 |
| 10 | 4.17E−05 | 10 | 9.00E−03 |
| 100 | 1.25E−06 | 100 | 1.00E−03 |
| 1000 | 1.21E−06 | 1000 | 4.00E−03 |
| 10000 | 1.43E−12 | 10000 | 3.00E−03 |

TABLE 2
Summary of combination effect with Compound A and
dexamethasone in myeloma cell line OPM-2, in
comparison to Compound B and dexamethasone.
OPM-2

| | CI of A + 100 nM | | CI of B + 100 nM |
|---|---|---|---|
| Cpd. A (nM) | Dex | Cpd. B (nM) | Dex |
| 0.0001 | 1.39E−01 | 0.0001 | 1.64E+00 |
| 0.001 | 5.00E−02 | 0.001 | 1.38E+00 |
| 0.01 | 4.90E−02 | 0.01 | 1.17E+00 |
| 0.1 | 1.50E−02 | 0.1 | 1.10E+00 |
| 1 | 1.10E−02 | 1 | 1.00E+00 |
| 10 | 3.00E−03 | 10 | 7.94E−01 |
| 100 | 7.00E−03 | 100 | 1.86E−01 |
| 1000 | 5.90E−02 | 1000 | 5.80E−02 |
| 10000 | 4.40E−02 | 10000 | 1.00E−03 |

TABLE 3
Summary of combination results at 50% growth inhibition between an
FGFR inhibitor and a glucocorticoid in multiple myeloma cell lines.

| | | | | Combination | |
|---|---|---|---|---|---|
| | | | | Index (CI) at | |
| | Chemical 1 | Chemical 2 | Synergy | 50% | Synergy |
| Cell Line | (mM) | (mM) | Score | inhibition | call |
| KMS-11 | Compound | Dexamethasone | 35 | 0.077 ± 0.001 | Very strong |
| | A (1.5) | (0.14) | | | synergism |
| OPM-2 | Compound | Dexamethasone | 29 | 0.034 ± 0.003 | Very strong |
| | A (0.03) | (0.30) | | | synergism |
| KMS-11 | Compound | Sicorten (0.01) | 10 | 0.220 ± 0.006 | Strong |
| | A (4.2) | | | | synergism |
| OPM-2 | Compound | Sicorten (0.03) | 17 | 0.282 ± 0.007 | Strong |
| | A (1.6) | | | | synergism |
| OPM-2 | Compound | Dexamethasone | 10 | 0.350 ± 0.010 | Synergism |
| | B (1.8) | (0.30) | | | |
| KMS-11 | Compound | Dexamethasone | 18 | 0.050 ± 0.004 | Very strong |
| | B (0.74) | (0.32) | | | synergism |
| OPM-2 | Compound | Sicorten (0.02) | 14 | 0.281 ± 0.005 | Strong |
| | B (10) | | | | synergism |
| KMS-11 | Compound | Sicorten (0.02) | 4 | 0.371 ± 0.003 | Synergism |
| | B (0.45) | | | | |

| Synergy Score | Best CI | Synergy call |
|---|---|---|
| ≧2 | <0.1 | Very strong synergism |
| ≧2 | 0.1-0.3 | Strong synergism |
| ≧2 | 0.3-0.7 | Synergism |
| ≧2 | 0.7-0.9 | Mild synergism |
| ≧2 | 0.9-1.1 | Additive |
| ≦1 | >1.1 | Antagonism |

We have demonstrated that several FGFR inhibitors and glucocorticoids have striking synergistic effects in inhibiting proliferation of multiple myeloma cell lines. The synergism has been observed over a wide range of concentrations of the FGFR inhibitors.

For example, the concentration of Compound A required to achieve 50% of inhibition of proliferation can be reduced by Dexamethasone by at least a million fold.

Methods:

Compound Preparation:

All FGFR inhibitors were desolved in DMSO as a 10 mM stock. Serial dilutions, as indicated in each figure, were made as 3× solutions in culture medium before adding to the cell cultures. Dexamethasone and halometasone were dissolved in 100% ethanol as a 10 millimol master stock. Serial dilutions, as indicated in each figure, were made as 3× solutions in culture medium. Cell lines, cell culture and treatment:

KMS-11 and OPM-2 cell lines can be purchased from HSRRB (Japan) and DSMZ (Germany), respectively. Early passage KMS-11 and OPM-2 cell lines were cultured in RPMI-1640 (ATCC Catalog#30-2001) supplemented with 10% FBS for 1 or 2 passages before treatment. Twenty thousand cells were seeded in each well of a 96-well plate and grew for 24 hours. Cells were then treated in triplicate with vehicle, an FGFR inhibitor (Compound A, B), a glucocorticoid (Dexamethasone or halometasone) alone or a combination at indicated concentrations in 5% CO₂ at 37 C for 72 hours. Viability was determined by CellTiter GLO (Promega, Cat#G755B) using identical method as described by the manufacturer. Data analysis:

Raw CellTiter GLO relative fluorescent unit (RFU) values were acquired using a microplate reader (PerkinElmer Precisely, Perkin Elmer Life and Analytical Sciences). Data analysis was performed using the Chalice software developed by CombinatoRx (Zalicus Inc., Cambridge, Mass., USA). Specifically, the Loewe Additivity (ADD) model was used as the combination reference in the EXAMPLES.

1. A combination of (a) a FGFR inhibitor and (b) a modulator of glucocorticoid receptor, wherein (a) and (b) are present in each case in free form, complex form or in the form of a pharmaceutically acceptable salt.

2. The combination of claim 1, wherein said FGFR inhibitor is selected from a group consisting of
(1) 3-(2,6-dichloro-3,5-dimethoxy-phenyl)-1-{6-[4-(4-ethyl-perpazin-1-yl)-phenylamino]pyrimidin-4-yl}-1-methyl urea;
(2) 4-amino-5-fluoro-3-[6-(4-methylpiperazin-1-yl)-1H-benzimidazol-2-yl]quinolin-2(1H)-one;
(3) (2R)-1-[4-(4-Fluoro-2-methyl-1H-indol-5-yloxy)-5-methyl-pyrrolo[2,1-f][1,2,4]triazin-6-yloxy]-propan-2-ol;
(4) (R)-N-(3-(3,5-Dimethoxy-phenyl)-ethyl)-1H-pyrazol-3-yl)-4-(3,4-dimethyl-piperazin-1-yl)benzamide;
(5) 1-tert-Butyl-3-[6-(3,5-dimethoxy-phenyl)-2-(4-diethylamino-butylamino)-pyrido[2,3-d]pyrimidin-7-yl]urea; and
(6) 3-(2,6-Dichloro-3,5-dimethoxy-phenyl)-1-methyl-1-[6-(4-piperazin-1-yl-phenylamino)-pyrimidin-4-yl]-urea.

3. The combination of claim 1, wherein said FGFR inhibitor is 3-(2,6-dichloro-3,5-dimethoxy-phenyl)-1-{6-[4-(4-ethyl-perpazin-1-yl)-phenylamino]-pyrimidin-4-yl}-1-methyl urea, in free form, complex form or in the form of a pharmaceutically acceptable salt.

4. The combination of claim 1, wherein said modulator of glucocorticoid receptor is dexamethasone or halometasone.

5. The combination of claim 1, wherein said FGFR inhibitor is 3-(2,6-dichloro-3,5-dimethoxy-phenyl)-1-{6-[4-(4-ethyl-perpazin-1-yl)-phenylamino]-pyrimidin-4-yl}-1-methyl urea, in free form, complex form or in the form of a pharmaceutically acceptable salt and said modulator of glucocorticoid receptor is dexamethasone.

6. The combination of claim 1 further comprises a pharmaceutically acceptable carrier.

7. The combination of claim 1 for simultaneous, separate or sequential use.

8. The combination of claim 1 being a fixed combination.

9. The combination of claim 8 further comprises a pharmaceutically acceptable carrier.

10. The combination of claim 1 for use in the treatment of haematological cancers.

11. The combination of claim 10, wherein said haematological cancer is multiple myeloma.

12. Use of the combination of claim 1, for the manufacture of a medicament for the treatment of haematological cancer.

13. Use of 3-(2,6-dichloro-3,5-dimethoxy-phenyl)-1-{6-[4-(4-ethyl-perpazin-1-yl)-phenylamino]-pyrimidin-4-yl}-1-methyl urea, in free form, complex form or in the form of a pharmaceutically acceptable salt, for the preparation of a medicament to be used in combination with a modulator of glucocorticoid receptor.

14. A commercial package comprising a combination according to claim 1, together with instructions for simultaneous, separate or sequential use thereof in the treatment of haematological cancers.

15. A method of treating haematological cancer, in a human patient, comprising administering to the human patient a combination according to claim 1.
